SEQUENCE LISTING (9 sequences)

SEQ ID NO: 1 - DNA, 24 bp, Artificial
  Oligonucleotide primer
agtgacaagaaataacaatacagg

SEQ ID NO: 2 - DNA, 24 bp, Artificial
  Oligonucleotide primer
cctgctttaagcactctaattttc

SEQ ID NO: 3 - DNA, 30 bp, Artificial
  Oligonucleotide primer
ggcgctacttcatataatataatgtttttt

SEQ ID NO: 4 - DNA, 32 bp, Artificial
  Oligonucleotide primer
ggcgctaattttaacttaaattggttaagaaa

SEQ ID NO: 5 - DNA, 25 bp, Artificial
  Oligonucleotide primer
tacttcatataatataatgtttttt

SEQ ID NO: 6 - DNA, 27 bp, Artificial
  Oligonucleotide primer
taattttaacttaaattggttaagaaa

SEQ ID NO: 7 - DNA, 259 bp, Cryptosporidium parvum
caatacaggactttttggttttgtaattggaatgagttaagtataaacccctttacaagtatcaattggagggcaagtctggtgccagcagccgcggtaattccagctccaatagcgtatattaaagttgttgcagttaaaaagctcgtagttggatttctgttaataatttatataaaatattttgatgaatatttatataatattaacataattcatattactatttttttttttagtatatgaaattttactttga

SEQ ID NO: 8 - DNA, 256 bp, Cryptosporidium parvum
caatacaggactttttggttttgtaattggaatgagttaagtataaacccctttacaagtatcaattggagggcaagtctggtgccagcagccgcggtaattccagctccaatagcgtatattaaagttgttgcagttaaaaagctcgtagttggatttctgttaataatttatataaaatattttgatgaatatttatataatattaacataattcatattactatatattttagtatatgaaattttactttga

SEQ ID NO: 9 - DNA, 256 bp, Cryptosporidium parvum
caatacaggactttttggttttgtaattggaatgagttaagtataaacccctttacaagtatcaattggagggcaagtctggtgccagcagccgcggtaattccagctccaatagcgtatattaaagttgttgcagttaaaaagctcgtagttggatttctgttaataatttatataaaatattttgatgaatatttatataatattaacataattcatattactatattttttagtatatgaaattttactttga

(19) United States
(12) Patent Application Publication
(10) Pub. No.: US 2006/0258855 A1
(43) Pub. Date: Nov. 16, 2006

(54) High resolution analysis of genetic variation within cryptosporidium parvum

(75) Inventors: Robin Gasser, Werribee (AU); Youssef Abs El-Osta, Werribee (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: Genetype Pty Ltd, Fitzroy (AU)

(21) Appl. No.: 10/525,029

(22) PCT Filed: Aug. 22, 2003
(86) PCT No.: PCT/AU03/01073

§ 371 Date: Oct. 31, 2005

(30) Foreign Application Priority Data
Aug. 22, 2002 (AU) 2002950977

(51) Int. Cl. C07H 21/02; C07H 21/04

(52) U.S. Cl. 536/23.1

(57) ABSTRACT

An oligonucleotide selected from at least one of the DNA sequences designated SEQ ID NO: 3, SEQ ID NO: 4, SEQ ID NO: 5, and/or SEQ ID NO: 6, or annealing equivalents thereof is described. Also disclosed is a method for the genotypic and subgenotypic identification of the genus Cryptosporidium in a sample.

### FIELD OF THE INVENTION

The present invention relates to detection of genetic variation within Cryptosporidium parvum and other members of this genus. More particularly, the invention relates to high resolution identification and genotyping of Cryptosporidium isolates using PCR primers directed against a nuclear ribosomal RNA locus.

### BACKGROUND OF THE INVENTION

Bibliographic details of the publications referred to herein are listed at the end of the description.

Commonly Used Abbreviations Are:

RNA, ribonucleic acid; DNA, deoxy ribonucleic acid; SSCP, single-strand conformation polymorphism analysis; DPGE, denaturing polyacrylamide gel electrophoresis; C. parvum, Cryptosporidium parvum; pSSU, ribosomal RNA locus in the small subunit gene of Cryptosporidium; pITS-2, ribosomal RNA locus in the second internal transcribed spacer of Cryptosporidium; PCR, polymerase chain reaction.

Methods based on the polymerase chain reaction (PCR) using various genetic markers have been employed in the identification of Cryptosporidium species, because of their ability to specifically amplify minute amounts of parasitic material. However, a range of prior art methods, such as PCR-based restriction fragment length polymorphism (RFLP), do not allow the accurate detection or display of genetic variation within and among isolates of Cryptosporidium.

There is a significant need for the identification of Cryptosporidium species and strains to assist in the elucidation of transmission patterns of Cryptosporidium and in the control of cryptosporidiosis.

### SUMMARY OF THE INVENTION

The subject of the invention, in its various aspects, allows for the identification of Cryptosporidium to the genotypic and subgenotypic levels. The invention therefore provides a foundation for the analysis of the genetic make-up of Cryptosporidium populations and epidemiological transmission, which assists in the control of cryptosporidiosis.

In one aspect, this invention provides at least one of the oligonucleotides of the DNA sequences designated SEQ ID NO: 3, SEQ ID NO: 4, SEQ ID NO: 5, and/or SEQ ID NO: 6, or annealing equivalents thereof.

In another aspect of this invention, there is provided a pair of PCR primers, one primer comprising at least 15 consecutive bases of the DNA sequence designated SEQ ID NO: 3 or an annealing equivalent thereof and a second primer comprising at least 15 consecutive bases of the DNA sequence designated SEQ ID NO: 4 or an annealing equivalent thereof, or at least 15 consecutive bases of the DNA sequence designated SEQ ID NO: 5 or an annealing equivalent thereof and a second primer comprising at least 15 consecutive bases of the DNA sequence designated SEQ ID NO: 6 or an annealing equivalent thereof.

The oligonucleotide sequences SEQ ID NO: 1-SEQ ID NO: 4 and, more particularly, the respective pairs of these primers described above, are adapted to amplify ribosomal DNA regions within the nuclear genome of Cryptosporidium, in particular the ITS-2 of Cryptosporidium.

In a further aspect of the present invention, there is provided a method for the genotypic and subgenotypic identification of Cryptosporidium in a sample, said method comprising the steps:
(a) providing a sample comprising genomic template DNA to be tested;
(b) providing a pair of PCR primers selected from the group consisting of:
   primers comprising at least 15 consecutive bases of the DNA sequence designated SEQ ID NO: 3 or annealing equivalents thereof and SEQ ID NO: 4 or annealing equivalents thereof, or at least 15 consecutive bases of the DNA sequence designated SEQ ID NO: 5 or annealing equivalents thereof and a second primer comprising at least 15 consecutive bases of the DNA sequence designated SEQ ID NO: 6 or an annealing equivalent thereof;
(c) amplifying by means of PCR a region of template DNA using said primer pair to produce one or more PCR products from the said sample, and thereafter analysing the PCR products so as to identify genotype and subgenotype Cryptosporidium in a sample.

Preferably, genomic template DNA of one or more Cryptosporidium standards of known genotype, and optionally known subgenotype, are also provided and amplified, so as to provide a standard during identification of Cryptosporidium genotype and subgenotype.

In the abovementioned embodiments, the first five bases 5′ of SEQ ID NO: 3 and SEQ ID NO: 4, which represent a “GC-tag”, are included in the primers to allow improved PCR amplification stringency and efficiency compared with primers of SEQ ID NO: 5 and SEQ ID NO: 6. These base pairs may be deleted and the present invention contemplates such primers and annealing equivalents thereof. SEQ ID NO: 5 and SEQ ID NO: 6, respectively, represent the “GC-tag”-deleted sequences SEQ ID NO: 3 and SEQ ID NO: 4.

Further aspects of this invention provide the use of any of the oliognucleotides as herein described or annealing equivalents thereof for the genotypic and subgenotypic characterisation of Cryptosporidium.

### DESCRIPTION OF THE FIGURES

The invention in its various aspects is illustrated in the accompanying examples with reference to the accompanying figures, in which:

FIG. 1 illustrates SSCP analysis of sequence variation in the two loci pSSU (A) and pITS-2 (B) within and among Cryptosporidium parvum samples originating from humans following recent foreign travel. A total of 103 samples were initially scanned by SSCP, and a sample subset is included on the present gel images. For pSSU, the first 19 samples (c2-c4) were identified as type-1 and the following 11 samples (c39-c42) as type-2. Samples c107 and c232 from humans from the UK were included as controls. For pITS-2, the first 8 samples (c40-c74) were identified as type-1 and the following 10 samples as type-2; the same two control samples were included as in panel A. Significant pITS-2 profile variation was detected within both types-i and -2. The level of sequence variation displayed was greater within and among samples representing type-2 compared with type-1. A total of 40 different bands (marked with white dashes) were excised (in the order top to bottom, left to right) from the pITS-2 SSCP gel and then subjected to sequencing.

FIG. 2 illustrates alignment of pSSU consensus sequences representing 20 C. parvum type-1 samples (codes c2, c4, c15, c20, c23, c31, c40, c41, c67, c107, c125, c128, c129, c136, c142, c14-9, c152, c154, c160 and c164) and 12 type-2 samples (codes c11, c32, c39, c42, c65, c118, c119, c122, c145, c159, c232 and c35). Of the type-2 samples, 11 samples were represented by the type-2.1 sequence, whereas sample c35 possessed the type-2.2 sequence. The type-2 sequences, which differed by a transversion at alignment position 230, could be delineated based on SSCP analysis (see FIG. 1). Nucleotide differences between Cryptosporidium parvum Types 1 and 2 are shown by (*). Summary of nucleotide differences: Type-1 vs type-2.1: 5 nt diffs (3 nt length diffs), Type-1 vs type-2.2: 4 nt diffs (3 nt length diffs), Type-2.1 vs type-2.2: 1 nt diff (no length diff).

FIG. 3 illustrates representative DPGE analysis of sequence variation in the two loci pSSU (A) and pITS-2 (B) within and among Cryptosporidium parvum samples from humans. Subsets of the 186 samples screened at both loci by SSCP, and representing the entire spectrum of sequence variation detected, have been included. For pSSU, the first 19 samples (c2-c4) were identified as type-1 and the following 11 samples (c39-c42) as type-2 (cf. FIG. 1). Samples c107 and c232 from humans from the UK were included as controls. For pITS-2, the first four samples (c76-c107) from a nursery outbreak in Middlesex (UK) and the next 11 samples (c40-c63) from humans following recent foreign travel were identified as type-1. Another 9 samples (c65-c42) from patients following overseas travel and 2 samples from a waterborne outbreaks in UK were identified as type-2. Significant pITS-2 profile variation was detected within both types-1 and -2. The level of sequence variation displayed within and among samples representing type-2 appeared greater than for type-1, in accordance with SSCP analysis (FIG. 1).

### SEQUENCE LISTING

Nucleotide sequences SEQ ID 1-6 are as follows:

| | |
|---|---|
| AGTGACAAGAAATAACAATACAGG | SEQ ID NO: 1 |
| CCTGCTTTAAGCACTCTAATTTTC | SEQ ID NO: 2 |
| GGCGCTACTTCATATAATATAATGTTTTTT | SEQ ID NO: 3 |
| GGCGCTAATTTTAACTTAAATTGGTTAAGAAA | SEQ ID NO: 4 |
| TACTTCATATAATATAATGTTTTTT | SEQ ID NO: 5 |
| TAATTTTAACTTAAATTGGTTAAGAAA | SEQ ID NO: 6 |

### DETAILED DESCRIPTION

The following is a description of preferred forms of the present invention provided in both general and specific terms in relation to the application and use of the novel oligonucleotides herein described for the identification of Cryptosporidium genotypes and sub genotypes

While the invention described herein is primarily directed to the use of the novel oligonucleotides as primers for the PCR amplfication from a range of species of Cryptosporidium, it will be readily appreciated that any of the oligonucleotides described herein may be used in alternative ways to the end of identifying strains of Cryptosporidium.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein “genus” is used to refer to a principal rank in the taxonomic hierarchy, falling below the family level and above the species level, and refers to the genus Cryptosporidium; “species” is used to refer to a fundamental rank in the taxonomic hierarchy falling below the genus level and indicating the limit of organisms able to interbreed; and “strain”, genotype (type) or subgenotype is used to refer to an operational taxonomic unit below the species level, which may indicate population variation within a species.

In a preferred form of the present invention, samples are of faecal origin, particularly containing Cryptosporidium oocysts, and are processed by conventional methods, such that the genomic DNA of any organism present in the sample is isolated. It will be appreciated that any method suitable for the isolation of genomic DNA of Cryptosporidium may be used, however, a preferred method is in the Examples.

Primarily, DNA isolated as abovementioned is subsequently used in the invention as a template in a PCR using either of the novel PCR primer pairs of the invention SEQ ID NO: 3 and SEQ ID NO: 4 and SEQ ID NO: 5 and SEQ ID NO: 6, or annealing equivalents thereof. The nucleotide sequences of each of these primers is provided in the examples and are also set out above. The primer pairs of the present invention enable Cryptosporidium and, more particularly, Cryptosporidium parvum (C. parvum) genotypes, and subgenotypes (or strains within genotypes) to be identified. Thus, each pair is applicable to any sample suspected of Cryptosporidium and the identification of a number of different species and strains within the genus Cryptosporidium.

According to the invention, a PCR primer (or, an oligonucleotide primer) is an oligonucleotide capable of specific hybridisation under particular PCR conditions to a region of the template DNA which has a sequence which is substantially complementary to the primer sequence and is able to prime the extension of DNA during PCR. It will be realised that a complementary sequence is capable of forming Watson-Crick bonds with its complement, in which adenine pairs with thymine or guanine pairs with cytosine. Each primer is typically used as a member of a primer pair, including a 5′ upstream primer which hybridizes with the 5′ end of the template DNA to be amplified and a 3′ downstream primer which hybridizes with the complement of the 3′ end of the template DNA to be amplified.

Those of ordinary skill in the art to which the invention relates will understand that the term “substantially complementary”, as used herein, means that the primer may not have 100% complementarity to its target template sequence but is capable of annealing thereto in a specific manner under appropriate PCR annealing conditions.

The primers of the present invention may be prepared using any number of conventional DNA synthesis methods. In the present case, the primers were manufactured and purchased commercially from GeneWorks Pty Ltd, PO Box 11, Rundle Mall, Adelaide, SA 5000, Australia.

In accordance with the preferred embodiment, optimal results have been obtained using primers which are identical in length and sequence to the primers SEQ ID NO: 3 and SEQ ID NO: 4 as abovementioned. However, a person of ordinary skill in the art will recognise that alterations may be made to the primers while still maintaining the genus-specificity of the PCR amplification and the efficacy of the present inventive diagnostic method. These hitherto unknown processes and sequences based thereon have particular utility in genotypic and subgenotypic identification of the genus Cryptosporidium.

Firstly, the length of the primers used may be modified. For example, the present invention contemplates that shorter primers containing at least 15 consecutive bases of the nucleotide sequences of these primers may be suitable. Similarly, the primers may be lengthened. The exact upper limit of the length of the primers can vary. However, typically primers will be less than or equal to 32 bases and preferably greater than or equal to 15 bases. By way of example, it is considered that primers referred to herein may be extended by up to 5-10 or more nucleotides at their 3′ and/or 5′ end. In addition, a non-complementary nucleotide sequence tract may also be attached to the 5′ end of each primer, thus increasing their length.

Secondly, the present invention contemplates minor changes (or conservative alterations) to the sequence of the primers which do not substantially alter their ability to anneal to their specific target DNA and subsequently prime extension during PCR. For example, any particular nucleotide, or plurality of nucleotides, of a primer may be substituted for alternative nucleotides, which may not allow for Watson-Crick base-pairing at the particular site of alteration on annealing of the primer to the template DNA during PCR, but nonetheless does not substantially affect the ability of the primer to prime extension during PCR. Such alternative primers may be referred to as “annealing equivalents” of the primers SEQ ID NO: 3 and-SEQ ID NO: 4 and variants thereof, as described herein. Such annealing equivalents will be at least 15 nucleotides in length and adapted to anneal to a target sequence under appropriate PCR annealing conditions. Generally, appropriate PCR annealing conditions for such annealing equivalents include the use of a PCR reaction mix or buffer having 1.5-7 mM MgCl₂. It is considered that annealing temperatures of between 45° C. and 55° C. may be appropriate for most annealing equivalents. By way of further exemplification, if 5 nucleotides within a particular primer sequence were altered in a manner described in this paragraph, and those alterations were spread centrally across the primer sequence, the preferred annealing temperature of that primer may decrease by approximately 5° C. A target sequence, as referred to in this paragraph, means a sequence being complementary to at least 15 consecutive bases of the sequence of the primers SEQ ID NO: 3, SEQ ID NO: 4, SEQ ID NO: 5 and SEQ ID NO: 6.

It will be appreciated that the usefulness of any alternative PCR primer sets designed around SEQ ID NO: 3 &-SEQ ID NO: 4, of the present invention, may be evaluated, at least notionally, using appropriate software and the pITS-2 and flanking DNA sequence information. Such software packages include, for example, PC Oligo5 (National Bioscience Inc) or Amplify (University of Wisconsin).

While the novel oligonucleotides or primers and variants thereof disclosed herein have been designed to enable identification of Cryptosporidium genotypes and subgenotypes, it will be appreciated that they may also be used, individually or in combination, for other applications. For example they may be used as molecular probes, or primers for alternative diagnostic techniques (such as LCR, ligase chain reaction or quantitative PCR). Those skilled in the art will readily appreciate the means by which such applications may be effected using standard methodology in the art.

In order that the PCR products may subsequently be detected, the primers are preferably end-labelled with [gammaγ-³³P]ATP. Alternatively, other means of labelling the PCR products may be utilised; for example, incorporation of [betaα³²P]dNTPs during PCR amplification. More preferably, non-radioactive labelling systems using digoxygenin, fluorescein, biotin and the like, may be employed. Also, unlabelled amplicons may be used for subsequent SSCP analysis using an SEA2000 electrophoresis apparatus (Elchrom, Switzerland). Such non-radioactive labelling systems are well known in the art and, accordingly, may be readily applied by a person of ordinary skill. It will be appreciated that the non-radioactive label may be used to end-label a particular primer or may be incorporated into the primer and/or PCR product.

Each PCR is generally carried out with at least one control sample or standard of known species identity. It will be appreciated that control samples containing more than one known species may be entertained. Negative controls in which no Cryptosporidium template DNA is present may also run against the samples. It will be appreciated that other standard controls routinely used in the art may also be run against the samples.

The Cryptosporidium genomic DNA of the control or standards of known Cryptosporidium species or genotypes may be purified in a like manner to the genomic DNA of unknown samples.

Amplification is conducted according to conventional procedures in the art to which this invention relates; for example, as described in U.S. Pat. No. 4,683,202. Preferably standard PCRs according to the invention include 0.1 μM-1 μM of each primer, 200 μM each dNTP, 1.5-7 mM MgCl₂, and 1U Taq DNA polymerase (Promega). Typically, each PCR is overlayed with mineral oil or the like to prevent evaporation of the reaction mix during cycling. PCR cycling is preferably run under the following conditions: an initial denaturation at a temperature of 94° C. for 5 min, followed by 30-35 cycles of denaturation at a temperature of 94° C. for approximately 15 to 30 seconds, annealing at a temperature of from 44° C. to 60° C. for 15 to 30 seconds and extension at a temperature of 72° C. for 15 to 30 seconds, followed by a final extension at 72° C. for 5 min.

It will be appreciated by those of ordinary skill in the art that the PCR conditions provided herein are merely exemplary and may be varied so as to optimise conditions where, for example, alternative PCR cyclers or DNA polymerases are used, where the quality of the template DNA differs, or where variations of the primers not specifically exemplified herein are used, without departing from the scope of the present invention. The PCR conditions may be altered or optimised by changing the concentration of the various constituents within the reaction and/or changing the constituents of the reaction, altering the number of amplification cycles, the denaturation, annealing or extension times or temperatures, or the quantity of template DNA, for example. Those of skill in the art will appreciate there are a number of other ways in which PCR conditions may be optimised to overcome variability between reactions. PCR conditions may be optimised by routine analysis based on the abovementioned factors which are commonly understood in the art.

It will be understood that, where not specifically exemplified herein, appropriate PCR annealing temperatures for any primer within the scope of the present invention may be derived from the calculated melting temperature of that primer. Such melting temperatures may be calculated using standard techniques. As will be understood by those of ordinary skill in the art to which this invention relates annealing temperatures may be above or below the melting temperature but generally an annealing temperature of approximately 5° C. above or below the calculated melting temperature of the primer may be suitable.

PCR products, of samples for identification and relevant control samples or standards of known identity, may be detected by electrophoretic separation. Electrophoretic techniques which are particularly sensitive to minor differences in PCR product size and/or sequence are preferred. For example, the techniques of SSCP (single-strand conformation polymorphism) and/or DPGE (denaturing polyacrylamide gel electrophoresis) are particularly suitable when conditions are optimised as they have the capacity to detect single base changes in sequence or variation in length by a single nucleotide between samples. In addition, these techniques are readily applicable to the screening of large numbers of samples.

SSCP analysis has been described (Orita et al., 1989). Generally, any particular PCR product (of usually ≦530 bp) may be separated as single-stranded molecules by electrophoresis in a non-denaturing polyacrylamide gel. The technique is based on the fact that a molecule of single-stranded DNA folds differently from another such molecule if it differs in sequence by a single base or more; differences in secondary and tertiary structures result in differences in mobility during electrophoresis.

Those of ordinary skill in the art to which this invention relates will understand that the tertiary structure of single-stranded DNA changes under different physical conditions, for example, temperature and ionic environment. As a result, the sensitivity of SSCP depends on these and many other such conditions, such as the length of the PCR product. In the case of the present invention, the following conditions have been found to be preferred, however, it will be appreciated that the conditions may be altered to take account of different laboratory conditions and equipment; 0.4 to 0.6×MDE (mutation detection enhancement; FMC BioProducts) containing 0.5 to 1.5×TBE and electrophoresis performed at 7-40 W for approximately 5-17 hours at 18° C. More specifically, the following conditions may be utilised in the invention:

| | Power | Time | Temperature |
|---|---|---|---|
| Gel | (W) | (hours) | (° C.) |
| 0.5 × MDE containing 0.6 × TBE | 7 | 17 | 18 |
| 0.4 × MDE containing 0.6 × TBE | 7 | 17 | 18 |
| 0.6 × MDE containing 0.6 × TBE | 20 | 5 | 18 |
| 0.6 × MDE containing 0.6 × TBE | 30 | 5 | 18 |
| 0.6 × MDE containing 0.6 × TBE | 40 | 5 | 18 |
| 0.6 × MDE containing 0.5 × TBE | 20 | 5 | 18 |
| 0.6 × MDE containing 1.0 × TBE | 30 | 5 | 18 |
| 0.6 × MDE containing 1.5 × TBE | 40 | 5 | 18 |

DPGE has previously been described and is well known in the art to which this invention relates. In DPGE, each strand of a DNA molecule is separated from its complementary strand and run on a polyacrylamide gel under denaturing conditions. Under such conditions, the two strands of any particular DNA molecule are prevented from re-hybridising to one another during electrophoresis such that individual strands will migrate separately within the gel. DPGE is a sensitive system which is capable of identifying differences in the length of any two DNA molecules to a single nucleotide.

In the case of DPGE, the following range of conditions are preferable, however, as with the SSCP conditions, they may be altered to take account of many other laboratory variables, without departing from the scope of the present invention; 0.4 mm thick gel between 4 and 6% polyacrylamide, containing 42% urea and 1×TBE, subjected to electrophoresis at between 30-60 W for approximately 3-4 hours at 40-45° C. More specifically, the following conditions may be utilised in the present invention:

| | Power | Time | Temperature |
|---|---|---|---|
| Gel (polyacrylamide) | (W) | (Hours) | (° C.) |
| 0.4 mm thick (6%) containing 42% | 50 | 4 | 40-45 |
| urea and 1 × TBE | | | |
| 0.4 mm thick (4%) containing 42% | 50 | 3 | 40-45 |
| urea and 1 × TBE | | | |
| 0.4 mm thick (5.5%) containing 42% | 50 | 3 | 40-45 |
| urea and 1 × TBE | | | |
| 0.4 mm thick (5%) containing 42% | 20 | 3 | 40-45 |
| urea and 1 × TBE | | | |
| 0.4 mm thick (5%) containing 42% | 30 | 3 | 40-45 |
| urea and 1 × TBE | | | |
| 0.4 mm thick (5%) containing 42% | 50 | 3 | 40-45 |
| urea and 1 × TBE | | | |

The present invention also contemplates both techniques being used in parallel, in order to gain a better understanding of the identity of the strain of Cryptosporidium parvum within a particular sample. Similarly, the present invention considers the use of other known oligonucleotide detection techniques, such as agarose gel electrophoresis, DNA sequencing and denaturing high performance liquid chromatography.

Following separation of PCR products via electrophoresis, gels may be processed according to standard techniques (for example, in the case of polyacrylamide gels, dried on to filter or blotting paper), and subjected to autoradiography for a time appropriate to be able to demonstrate the position of the bands on a gel.

The methodology of the present invention is adaptable to an automated (fluorescence-based) electrophoretic system; for example, and Applied BioSystems (ABI) automated sequencing apparatus coupled with a thermocontrol system and appropriate computer hard- and software. In this way, specific “fingerprints” may be recorded, stored (for protocol and reporting purposes) and compared against standard samples of known subgenotypic status. Such electrophoretic methods are standard procedures commonly used and understood by those of general skill in the art.

It will be appreciated that where an automated system is used in the present invention that standard conditions generally applicable to manual SSCP and DPGE, as described and exemplified herein, may need to be varied in order to accommodate the particular automated system utilised. Appropriate conditions may be easily determined from manufacturer's instructions and routine optimisation assays according thereto and as are commonly used in the art.

### EXAMPLES

Aspects of the present invention are described in the illustrative, non-limiting examples hereafter.

1. Materials and Methods

1.1. Isolates, DNA Isolation and Enzymatic Amplification

Cryptosporidium oocyst isolates were obtained from Dr Rachel Chalmers, PHLS Cryptosporidium Reference Unit, Singleton Hospital, Swansea, Wales, UK. The oocyst isolates are from humans with clinical cryptosporidiosis reported to be contracted during recent international travel (n=103) or during outbreaks (n=83) in the United Kingdom (Tables 1 and 2). Oocysts were purified, and genomic DNA isolated using standard approaches (see Chalmers et al., 2002). Two regions of the nuclear genome were PCR-amplified separately using oligonucleotide primers 18SiF (forward, 5′-AGTGACAAGAAATAACAATACAGG-3′) and 18SiR (reverse, 5′-CCTGCTTTAAGCACTCTAATTTTC-3′) (˜300 bp region of the SSU rRNA gene, designated pSSU; Khramtsov et al., 1995; Morgan et al., 1997; Gasser et al., 2001a), and primers YA56F (forward: 5′-GGCGCTACTTCATATAATATAATGTTTTTT-3′ (SEQ ID NO: 3)) and YA54R (reverse: 5′-GGCGCTAATTTTAACTTAAATTGGTTAAGAAA-3′ (SEQ ID NO: 4)) (˜230 bp region of the ITS-2, designated pITS-2; cf. Morgan et al., 1999b). A “GC-tag” (first five bases 5′) was included in both primers YA56F and YA54R to allow improved amplification stringency and efficiency (unpublished results). Primers were end-labelled with ³³P-ATP (NEN, DuPont) using T4 kinase (Promega). Primers which do not include these GC-tags are respectively SEQ ID NO: 5 and SEQ ED NO: 6.

PCR amplification was performed in 50 μl volumes using 25 pmol of each primer, 250 μM of each dNTP, 3 mM MgCl₂ and 1 U Taq polymerase (Promega, Madison, Wis., USA) under the following conditions: after an initial denaturation at 94° C. for 5 min, reactions were subjected to 30 cycles of 94° C. for 30 s (denaturation), 55° C. (pSSU) or 50° C. (pITS-2) for 30 s (annealing) and 72° C. for 30 s (extension), followed by a final extension at 72° C. for 5 min in a 480 thermocycler (Perkin Elmer Cetus, Norwalk, Conn., USA). The amount of template added to the PCR reaction was usually 1 pg to 20 ng. Control samples without DNA were included in each PCR run. To test the specificity of the PCR, 20 ‘negative’ control samples, including DNA from faeces or blood from a human without any evidence of parasitic infection, and DNA from the bacteria Escherichia coli, Lactobacillus acidophilus and L. gasseri; the yeast, Saccharomyces cerevisiae, protozoan parasites, Giardia duodenalis and Entamoeba histolytica; and helminth parasites, Schistosoma japonicum, S. mansoni, Hymenolepis nana, Taenia saginata, T. solium, Ascaris sp., Ancylostoma caninum, Necator americanus, Strongyloides stercoralis, Oesophagostomum bifurcum and Trichuris trichiura) were subjected to the same amplification procedure (using both primer sets). Two C. parvum DNA sample (designated c107 and c232 and categorized previously as type-1 and type-2, respectively) were included as ‘positive’ controls.

After thermocycling, individual amplicons were mixed with an equal volume of loading buffer (10 mM NaOH, 95% formamide, 0.05% of both bromophenol blue and xylene cyanole) and the intensity of selected samples verified on ethidium bromide-stained 2.5% agarose gels using TBE (65 mM Tris-HCl, 27 mM boric acid, 1 mM EDTA, pH 9; Bio-Rad, Richmond, Calif., USA) as the buffer and PhiX174-HaeIII (Promega, Wis., USA) as a size marker.

1.2 Electrophoresis

Radiolabelled amplicons were denatured at 95° C. for 5 min and snap-cooled on a freeze block (−20° C.) for 5 min prior to SSCP or denaturing polyacrylamide gel electrophoresis (DPGE) in a conventional sequencing rig (S2, Life Technologies, USA) (see Zhu and Gasser, 1998; Woods et al., 2000). SSCP analysis was performed in 0.4 mm-thick, 0.6× mutation detection enhancement gels (MDE; FMC BioProducts, Rockland, Me., USA), cast according to the manufacturer's protocol. Gels were poured (inserting a 60 tooth well-comb; well-width of 3 mm), allowed to polymerise at 24° C. for 70 min and subjected to electrophoresis in an air-conditioned room (18° C. constant) using TBE as the buffer. Gels were pre-run at 30 W (constant) for 20 min (until the gel had reached a temperature of ˜24° C.), and samples (1.7 μl) were loaded into wells and subjected to electrophoresis under the same conditions for 5 h. DPGE was carried out in 0.4 mm thick 5% polyacrylamide gel containing 42% urea. Samples (3 μl each) were loaded into shark-tooth comb wells (4 mm wide) and subjected to electrophoresis at 50 W for 3 h at 24° C. (achieving a glass plate temperature of ˜39-40° C.) using TBE. For both electrophoretic procedures, gels were dried on to 3MM filter paper (Whatman) and usually subjected to autoradiography for 24 h using Curix-Blue film (Agfa). Electrophoretic profiles obtained using each of the electrophoretic approaches were demonstrated to be reproducible on different days using amplicons produced on different days (results not shown).

1.3 DNA Sequencing

Selected pSSU amplicons were purified over Wizard PCR Preps columns (Promega, Wis., USA) and eluted with 30 μl H₂O and then subjected to automated cycle-sequencing (BigDye chemistry, ABI) using the same primers as for PCR. SSCP bands representing selected pITS-2 amplicons were enriched as described previously (Gasser and Monti, 1997) and then sequenced via cloning. In brief, after autoradiographic exposure of gels, selected SSCP bands were excised with a scalpel, suspended in 50 μl water and incubated at 4° C. for 2 h. Then, 2 μl of this suspension were added to 98 μl water and 1-2 μl thereof subjected to the PCR (using the same primer set and conditions as used for primary amplification). An aliquot of column-purified amplicon (˜100 ng) was then cloned into the pGEM-T Easy vector system I (Promega, Wis., USA). Clones were isolated, and inserts amplified and subsequently sequenced employing the vector primer SP6 or T7 (Promega, Wis., USA). Nucleotide sequences were subjected to BLASTX and BLASTP similarity searches of the non-redundant GenBank database (NCBI Basic BLAST; http://www.ncbi.nlm.nih.gov/BLAST/).

3. Results and Discussion

The specificity of the primer sets 18SiF-18SiR (pSSU) and YA56F-YA54R (pITS-2) and the PCR conditions for the amplification of C. parvum pSSU and pITS-2 were assessed using a range of control DNA samples, followed by electrophoretic analysis and autoradiography. DNA samples prepared from human blood or faeces (from a person with no evidence of parasitic infection) and 17 additional samples from a range of prokaryotic (bacteria) or eukaryotic organisms (protozoa, trematodes, cestodes and nematodes), developmental stages of which can occur in the intestinal tracts of humans, were also subjected to PCR. Using ˜0.5-5 ng of template per PCR reaction, appropriately sized products were amplified from samples c107 and c232 (positive controls) and detected by electrophoresis, whereas no bands were detectable for any of the other 19 control samples tested (not shown).

The sensitivity of the PCR was determined by titration of genomic DNA representing samples c107 and c232, followed by SSCP analysis (not shown). The lowest amounts of C. parvum DNA detectable by PCR amplification using the primer set 18SiF-18SiR (pSSU) or YA56F-YA54R (pITS-2) and subsequent analysis was estimated at 1-2 pg, which is comparable with a previous study of other members within the Eimeriidae (Woods et al., 2000).

Having demonstrated the specificity and sensitivity of the PCR and the conditions, 186 C. parvum samples from humans representing sporadic cases of cryptosporidiosis following recent overseas travel (n=103) or cases linked to cryptosporidiosis outbreaks in the UK (n=83) were subjected to PCR. As expected, amplicons were ˜300 bp (pSSU) or 230 bp (pITS-2) in size on agarose gels, and no unequivocal size variation was detectable among any of the amplicons representing each of the rDNA loci (not shown). Partial sequencing of selected pSSU and pITS-2 amplicons and comparison of sequence data with entries in current databases verified their identity. All 186 samples were then subjected to high resolution electrophoretic analysis.

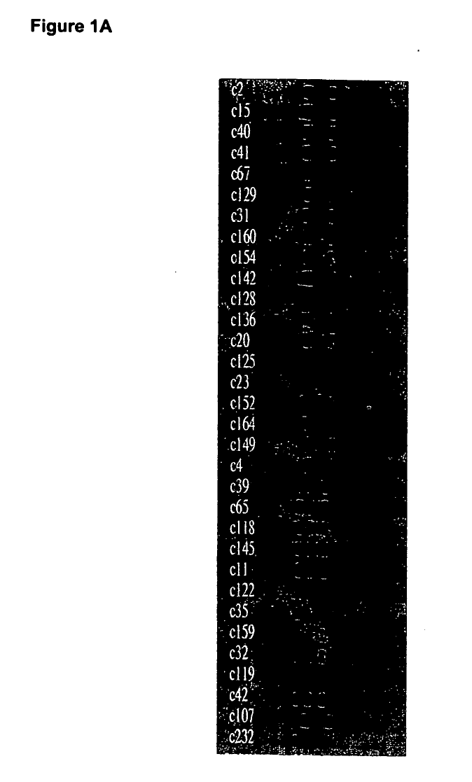

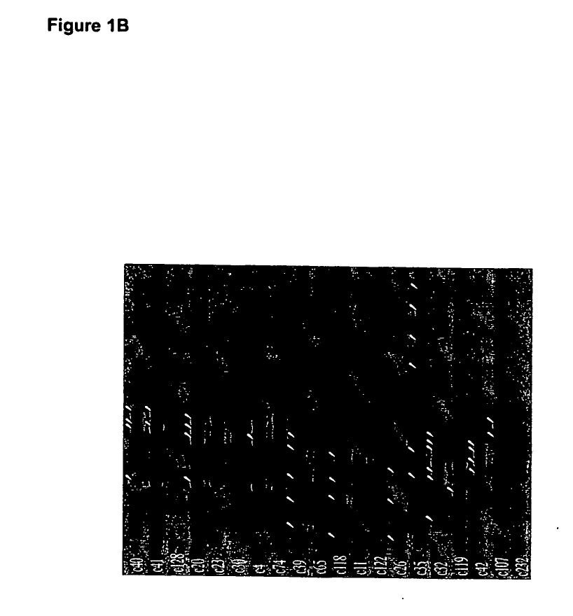

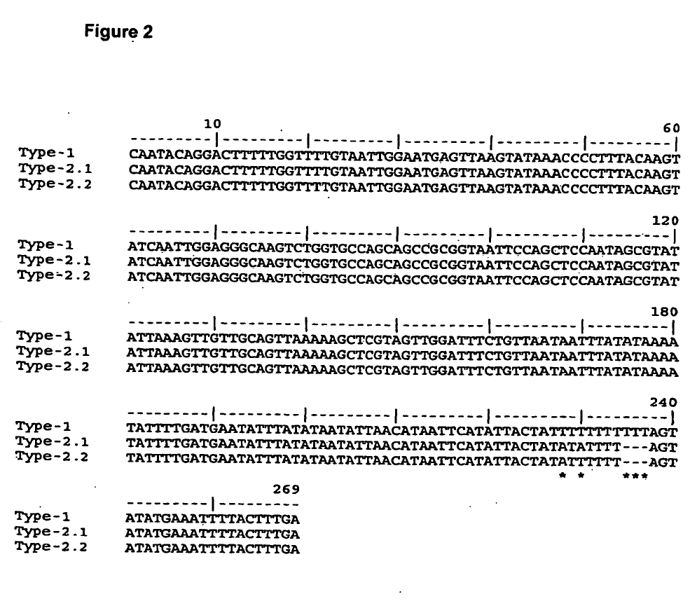

Results of the SSCP analyses of the pSSU and pITS-2 amplicons are shown in Tables 1 and 2, and representative electrophoretic gels are shown in FIG. 1. In the SSCP analysis of pSSU, 89 of the 103 (86.4%) ‘foreign traveller’ samples could be identified as type-1, whereas 14 samples (13.6%) were identified as type-2. No variation in SSCP profiles was detectable among any of the samples representing type-1, and only one type-2 sample (c35) differed slightly in profile (subtle upward shift) from all other samples of that genotype (see FIG. 1). Sequencing of representative samples (codes c11, c32, c35, c39, c42, c65, c118, c119, c122, c145, c159 and c232; see FIG. 1) revealed 1 nucleotide difference (A<->T transversion) at alignment position 230 between sample c35 and all other type-2 samples sequenced (see FIG. 2). BLAST analysis revealed a perfect match of the c35 sequence (over alignment positions 142-269) with that linked to accession no. AF133842 (Pieniazek et al., unpublished), it thus being distinct from the vast majority of other type-2 sequences reported to date. In contrast, no fixed nucleotide difference was detected in the pSSU among any of the type-1 samples sequenced (codes c2, c4, c15, c20, c23, c31, c40, c41, c67, c125, c128, c129, c136, c142, c149, c154, c152, c160 and c164) (FIG. 2), the sequences of which were in accordance with a range of sequences representing type-1 in current databases (e.g., accession no. AF093491). Irrespective of the mutation detected (at alignment position 230; FIG. 2) within type-2 by SSCP analysis of the pSSU locus, a distinct difference in the position of bands (relating to a 4-5 bp difference over 269 bp, excluding primers) enabled the unequivocal identification of and delineation between C. parvum type-1 and type-2 by SSCP analysis (FIG. 1). Also for ‘outbreak’ samples (Table 2), unequivocal differentiation of type-1 (n=21; child day-care nursery outbreak in Middlesex, UK) from type-2 (n=62; waterborne outbreaks in Clitheroe and Cleethorpe, UK) was achieved.

While no or a low level of profile variation was detectable within each of the two genotypes in the SSCP analysis of pSSU (FIG. 1), a total of 13 and 10 different pITS-2 profiles (˜4-12 bands per profile) were displayed for all 186 samples representing C. parvum type-1 and type-2, respectively (Tables 1 and 2). A representative SSCP gel displaying some of these pITS-2 profiles is shown in FIG. 1. More profile variation was displayed among type-2 than type-1 samples. In order to verify that the bands within individual SSCP profiles did indeed represent the ITS-2 of C. parvum, 40 different bands (representing the spectrum of sequence variability) (indicated in FIG. 2) were excised, and then subjected to PCR-enrichment and sequencing. The sequences derived from individual bands (nos. 1-11 representing type-1; nos. 12-40 type-2) were compared with respective ITS sequences (accession nos. AF015774, AF093008, AF093012 and/or AF040725) available in sequence databases. Sequence identities of ˜76-99% for type-1 and ˜75-99% for type-2 were recorded, thus providing clear support for the identity of individual bands as ITS-2. Pairwise comparison among the band sequences representing each of the genotypes revealed differences ranging from ˜62-99% (type-1) and ˜45-99% (type-2).

For ‘foreign traveller’ samples (Table 1), pITS-2 SSCP profiles 1A and 1B were represented by 34 (38.2%) and 35 (39.3%) of the 89 C. parvum type-1 samples analysed, whereas the other nine profiles (1C-1K) were represented by 1-6 (1.1-6.7%) of those samples (Table 1). For type-1 samples, profiles 1A and 1B displayed the widest geographical distribution, followed by profiles 1G, 1C, 1F and 1J, whereas profiles 1D, 1E, 1H, 1I and 1K were each unique to a particular geographical region. Although the sample size representing type-2 was ˜16% of that representing type-1, the SSCP analysis of pITS-2 amplicons revealed 9 distinct profiles (2A-2I) (Table 1). For these samples, profile D had the widest geographical distribution and profiles 2A-2C occurred on the Iberian Peninsula, whereas profiles 2E-2I were each unique to a particular location. Profile 2F (representing sample c35) was the most distinct with respect to all other type-2 samples, in accordance with the SSCP and sequence analyses of the pSSU locus for this sample. Overall, the SSCP results for the ‘foreign traveller’ samples would suggest some association between some SSCP profiles and geographical orgin, in spite of a bias in the numbers of samples from particular countries or geographical regions. For ‘outbreak’ samples (Table 2), no variation in profiles was detected among the 62 samples representing the waterborne epidemics in the UK (type-2), whereas four different pITS-2 SSCP profiles (1A, 1B, 1L and 1M) were detected among the 21 samples representing the Middlesex nursery outbreak. These latter findings would suggest that at least four different sources of C. parvum had contributed to the nursery outbreak. In spite of ‘intragenotypic’ profile variability detected within both ‘foreign traveller’ and ‘outbreak’ sample groups, the two genotypes of C. parvum could be distinguished based on the pITS-2 SSCP profiles (FIG. 1). Importantly, the genotypic categorization of samples as type-1 or as type-2 based on SSCP analysis of pITS-2 was in accordance with the results achieved using the pSSU locus.

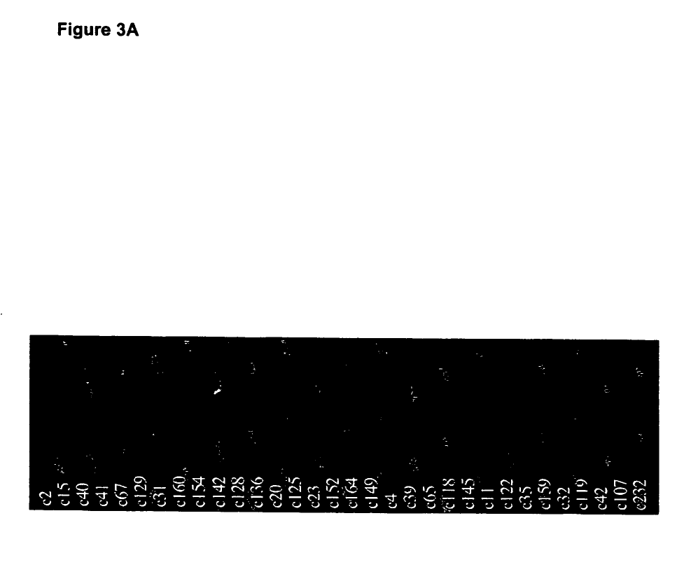

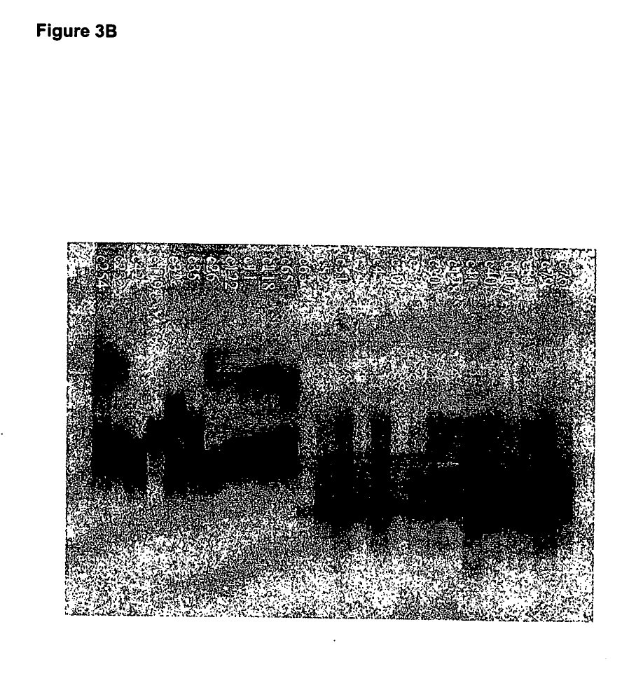

After having conducted the SSCP analyses and confirmed sequence/length variation in both the pSSU and pITS-2 loci between and/or within genotypes (FIGS. 1-3), we aimed at decreasing the electrophoretic running time required for analysis of amplicons for routine diagnosis. It was therefore evaluated whether reliable differentiation between type-1 and type-2 of C. parvum could also be achieved by DPGE using either or both of the ribosomal loci. It was also assessed whether the intragenotypic variability detectable in the pITS-2 locus by SSCP analysis could be displayed reliably (see FIG. 3). DPGE analysis of the samples representing the entire spectrum of variability (for the 186 samples) revealed the same genotypic identification and a similar subgenotypic classification as the SSCP analysis (FIG. 3). While the subgenotypic classification of the type-2 samples by DPGE was the same as for SSCP analysis, ˜30% less profile variation (9 instead of 13 profiles) was detectable among all type-1 samples. These results reinforced the superior ability of SSCP to detect subtle sequence variation among molecules of the same length within an amplicon (cf. Gasser and Chilton, 2001; Woods et al., 2000) and reflected the SSCP result of increased sequence variability in the pITS-2 within and among type-2 samples compared with type-1.

Significant population variation (i.e., sequence and/or length variation) in the pITS-2 was detected within each of the two C. parvum genotypes, which contrasts the original belief of limited intragenotypic variability (references cited in Gasser and O'Donoghue, 1999; Fayer et al., 2000) and supports a number of more recent studies (e.g., Aiello et al., 1999; Caccio et al., 2000, 2001; Guyot et al., 2001; Ong et al., 2002). For example, a recent investigation (Caccio et al., 2000) using microsatellite analysis of 94 C. parvum samples from humans and animals revealed two subgenotypes within type-1 samples and four subgenotypes within type-2. Some of the subgenotypes had a broad geographical distribution, whereas others were limited to specific locations. In another study using nine microsatellite loci (Aiello et al., 1999), two distinct subgenotypes were identified within each type-1 and type-2. In addition to its significance for investigating population genetic structures and possibly transmission patterns of C. parvum, the ability to accurately display sequence heterogeneity in the ITS may be of fundamental relevance for studying molecular evolutionary processes and inheritance in C. parvum. A detailed study of C. parvum (see Le Blancq et al., 1997) demonstrated that there are (at least) five copies of the rDNA unit per haploid genome, which are not organised in a conventional head-to-tail tandem array. There is evidence that the rDNA units are dispersed (as single copies) to at least three different chromosomes. Two structurally distinct variants of rDNA unit were detected, namely variant-A (four copies) and variant-B (one copy), and the most significant sequence variability between the two variants related to the ITS. While the magnitude of sequence divergence in the ITS region of C. parvum appears to be similar to that in the related apicomplexan parasite, Plasmodium berghei (see Dame et al., 1984), the functional significance of the two variants of rDNA in C. parvum is presently unknown, although in the malarial parasite, the different sets of rRNA genes give rise to structurally distinct, stage-specific ribosomes (Gunderson et al., 1987; McCutchan et al., 1995). It is also unclear whether such heterogeneity relates to sequence variation within or among individual oocysts of C. parvum within an isolate. A micromanipulation, PCR-based SSCP approach (cf. Sturbaum et al., 2001) may be useful to address this question through the analysis of single Cryptosporidium oocysts.

The relatively high degree of sequence heterogeneity in the ITS-2 (which is present at low copy number and as single copies on different chromosomes) indicates that sequence homogenisation (due to ‘concerted evolution’; Elder and Turner, 1995) is less effective in C. parvum (cf. FIG. 1) compared with some other organisms (species) with a high copy numbers (on a single locus) organised in tandem arrays, where sequence homogeneity is maintained by intrachromosomal exchange processes (see Schlötterer and Tautz; Gasser et al., 1998, 2001b). The sequence heterogeneity in the ITS of some apicomplexan parasites appears to be constrained by selection to maintain distinct sets of rRNA genes (see McCutchan et al., 1995; Woods et al., 2000). While intraisolate (or intraorganismal) variation can represent a practical problem for genetic analyses of oocyst DNA isolates using some PCR approaches (see Gasser, 1997), it presents a distinct advantage for the detection of population variation within Cryptosporidium by mutation scanning, as demonstrated clearly herein. Although the significance of the subgenotypic variability in the pITS-2 is currently unknown, some subgenotypes represented by SSCP profiles 1A, 1B, 1G and 2D (Table 1) showed a relatively broad geographical distribution, whereas others were restricted to particular regions. Further study needs to establish whether there is a definite link to the geographical origin of an isolate. If this is indeed the case, this would have important implications for the “tracking” of original sources of infection.

The results of the present study demonstrate the applicability of both SSCP and DPGE to screen C. parvum DNA samples for genetic variation and to categorize them to both the genotypic and subgenotypic levels. These electrophoretic methods have significant advantages over some other molecular-diagnostic approaches applied to Cryptosporidium. In contrast to some PCR-based approaches which rely on the genotypic identification of Cryptosporidium based solely on the detection of bands of a particular sizes on agarose gels (providing a phenotypic rather than a genotypic read-out), SSCP enables a detailed, qualitative analysis of amplicons for both length and sequence variability. The present SSCP was able to detect the transversional event (T<->A) (alignment position 230; FIG. 2) in the pSSU of type-2 samples, demonstrating its high mutation detection rate. This information, together with preliminary findings for C. felis, C. serpentis and C. baileyi (see Gasser et al., 2001a), reinforces that SSCP analysis of pSSU should enable the specific identification of all 10 currently recognised species of Cryptosporidium (cf. Fayer et al., 2000), although this proposal still requires testing. The concordance between results for pSSU and pITS-2, and the ability to directly ‘fingerprint’ intragenotypic nucleotide variability in the ITS-2 region, clearly reinforces the value of mutation scanning for detecting population variation within Cryptosporidium species. Using both SSCP and DPGE, large sample sizes can be scanned directly for genetic variation circumventing the need for DNA sequence analysis (in the first instance), which reduces time and labour. The approaches are also considerably less expensive than, for example, real-time PCR because they use conventional equipment readily available in most molecular biology laboratories. Therefore, the present tools should be of particular value for the diagnosis and monitoring of cryptosporidiosis outbreaks as well as for studying the epidemiology and the genetic make-up of Cryptosporidium populations. Future work will involve the automation of electrophoretic analysis which will increase throughput capability, and should simplify the recording, storage and comparison of profile data. Similar approaches will be applicable to a range of other pathogens of socio-economic importance, provided suitable genetic loci are employed.

### REFERENCES

Aiello A E, Xiao L, Limor J R, Liu C, Abrahamsen M S, Lal A A (1999) J Eukaryot
Microbiol 46, 46S-47S.
Carraway M, Tzipori S, Widmer G (1996) Appl Environ Microbiol 62, 712-716.
Caccio S, Homan W, Camilli R, Traldi G, Kortbeek T, Pozio E (2000) Parasitology 120, 237-244.
Caccio S, Spano F, Pozio E (2001) Int J Parasitol 31, 1082-1086.
Chalmers R M, Elwin K, Reilly W J, Irvine H, Thomas A L, Hunter P R (2002) Int J Parasitol 32, 21-26.
Dame J B, Sullivan M, McCutchan T F (1984) Nucleic Acids Res 12, 5943-5952.
Elder J F, Turner B J (1995) Quart Rev Biol 70, 297-320.
Fayer R, Morgan U, Upton S J (2000) Int J Parasitol 30, 1305-1322.
Gasser R B (1997) Int J Parasitol 27, 1449-1463.
Gasser R B, Chilton N B (2001) Vet Parasitol 101, 201-213.
Gasser R B, Monti J R (1997) Mol Cell Probes 11, 201-209.
Gasser R B, O'Donoghue P (1999) Int J Parasitol 29, 1379-1413.
Gasser R B, Zhu X Q, Chilton N B, Newton L A, Nedergaard T, Guldberg P (1998) Electrophoresis 19, 2391-2395.
Gasser R B, Zhu X Q, Caccio S, Chalmers R, Widmer G, Morgan U, Thompson R C A, Pozio E, Browning G F (2001a) Electrophoresis 22, 433-437.
Gasser R B, Zhu X Q, Beveridge I, Chilton N B (2001b) Electrophoresis 22, 1076-1085.
Gunderson J H, Sogin M L, Wollett G, Hollingdale M, De La Cruz V F, Waters A P, McCutchan T F (1987) Science 238, 933-937
Guyot K, Follet-Dumoulin A, LeLievre E, Sarfati C, Rabodonirina M, Nevez G, Cailliez J C, Camus D, Dei-Cas E (2001) J Clin Microbiol 39, 3472-3480.
Khramtsov, N. V., Tilley, M., Blunt, D. S., Montelone, B. A., Upton, S. J., J. Euk. Microbiol 1995, 42, 416-422.
Le Blancq S M, Khramstov N V, Zmani F, Upton S, Woo T W (1997) Mol Biochem Parasitol 90, 463-478.
McCutchan T F, Li J, McConkey G A, Rogers M J, Waters A P (1995) Parastol. Today 11, 134-138.
Morgan U M, Constantine C C, Forbes D A, Thompson R C A (1997) J Parasitol 83, 825-830.
Morgan U M, Xiao L, Fayer R, Lal A A, Thompson R C A (1999a) Variation in Cryptosporidium: towards a taxonomic revision of the genus. Int J Parasitol 29, 1733-1751.
Morgan U M, Deplazes P, Forbes D A, Spano F, Hertzberg H, Sargent K D, Elliot A, Thompson R C A (1999b) Parasitology 118, 49-58.
Narayanaswami G, Taylor P D (2001) Genetic Testing 5, 9-16.
O'Donoghue P J (1995) Int J Parasitol 25, 139-195.
Ong C S, Eisler D L, Alikhani A, Fung V W., Tomblin J, Bowie W R, Isaac-Renton J L (2002) Emerg Infect Dis 8(3):263-8, 2002 March.
Quintero-Betancourt W, Peele E R, Rose J B (2002) J Microbiol Meth 49, 209-224.
Schlötterer C, Tautz D (1994) Curr Biol 4, 777-783.
Sturbaum G D, Reed C, Hoover P J, Jost B H, Marshall M M, Sterling C R (2001) Appl Environ Microbiol 67, 2665-2668.
Widmer G (1998) Adv Parasitol 40, 223-239.
Woods W G, Richards D G, Whithear K G, Anderson G R, Jorgensen W, Gasser R B (2000) Electrophoresis 21, 3558-3563.
Xiao L, Sulaiman I, Fayer R, Lal A A (1998) Mem Inst Oswaldo Cruz (Rio de Janeiro) 93, 687-692.

Zhu X Q, Gasser R B (1998) Electrophoresis 19, 1366-1373.

TABLE 1
Results from SSCP analysis of pITS-2 amplicons produced from
Cryptosporidium parvum (type-1 or 2) oocyst DNA originating from
faecal samples from humans with recent travel to different countries
or continents and returning to the United Kingdom

| SSCP profile | | Travel destination (No. of | No. of |
|---|---|---|---|
| pSSU | pITS-2 | samples analysed) | samples |
| 1 | 1A | Spain (19), Greece (2), Cyprus (3), | 34 |
| | | Egypt (1), Pakistan (4), USA (1), | |
| | | Jamaica (1), Cuba (1), Peru (1), | |
| | | Mexico (1) | |
| 1 | 1B | Denmark (1), France (2), Spain (18), | 35 |
| | | Cyprus (2), Greece (2), Turkey (3), | |
| | | Pakistan (3), India (1), Mexico (2), | |
| | | Carribean Islands (1) | |
| 1 | 1C | Kenya (1), Gambia (1) | 2 |
| 1 | 1D | Kenya (1) | 1 |
| 1 | 1E | Pakistan (1) | 1 |
| 1 | 1F | Pakistan (2), USA (1) | 3 |
| 1 | 1G | Nigeria (1), Pakistan (2), India (1), | 6 |
| | | Caicos Islands (1), Equador (1) | |
| 1 | 1H | Spain (2) | 2 |
| 1 | 1I | Jamaica (1) | 1 |
| 1 | 1J | Spain (2), Greece (1) | 3 |
| 1 | 1K | Menorca (1) | 1 |
| 2.1 | 2A | Spain (3) | 3 |
| 2.1 | 2B | Spain (1) | 1 |
| 2.1 | 2C | Portugal (2) | 2 |
| 2.1 | 2D | Portugal (1), Uganda (1), | 3 |
| | | Saudi Arabia (1) | |
| 2.1 | 2E | Africa (1) | 1 |
| 2.2 | 2F | Cyprus (1) | 1 |
| 2.1 | 2G | Turkey (1) | 1 |
| 2.1 | 2H | Pakistan (1) | 1 |
| 2.1 | 2I | Mexico (1) | 1 |
| | | | [Total 103] |

TABLE 2
Results from SSCP analysis of pITS-2 amplicons produced from
Cryptosporidium parvum (type-1 or 2) oocyst DNA samples originating
from faecal samples from humans associated with two different
cryptosporidiosis outbreaks in the United Kingdom

| SSCP profile | | | No. of |
|---|---|---|---|
| pSSU | pITS-2 | Description | samples |
| 1 | 1A | Nursery outbreak in Middlesex | 13 |
| 1 | 1B | Nursery outbreak in Middlesex | 1 |
| 1 | 1L | Nursery outbreak in Middlesex | 6 |
| 1 | 1M | Nursery outbreak in Middlesex | 1 |
| 2 | 2J | Waterborne outbreak in Clitheroe | 39 |
| | | Waterborne outbreak in Cleethorpe | 23 |
| | | | [Total 83] |

1. An oligonucleotide selected from at least one of the DNA sequences designated SEQ ID NO: 3, SEQ ID NO: 4, SEQ ID NO: 5, and/or SEQ ID NO: 6, or annealing equivalents thereof.

2. A oligonucleotide according to claim 1 which comprises a pair of PCR primers for identification of the genus Cryptosporidium to the genotypic and subgenotypic level, comprising at least 15 consecutive bases of the DNA sequence designated SEQ ID NO: 3 or an annealing equivalent thereof and a second primer comprising at least 15 consecutive bases of the DNA sequence designated SEQ ID NO: 4 or an annealing equivalent thereof, or at least 15 consecutive bases of the DNA sequence designated SEQ ID NO: 5 or an annealing equivalent thereof and a second primer comprising at least 15 consecutive bases of the DNA sequence designated SEQ ID NO: 6 or an annealing equivalent thereof.

3. An oligonucleotide according to claim 1 wherein said annealing equivalents comprise at least 15 nucleotides in length, and are adapted to anneal to a target sequence which is complementary to at least 15 consecutive bases of a primer selected from SEQ ID NO: 3, SEQ ID NO: 4, SEQ ID NO: 5 or SEQ ID NO: 6 at a temperature of 45 to 55° C. in a buffer containing 1.5-7 mM MgCl₂.

4. A method for the genotypic and subgenotypic identification of the genus Cryptosporidium in a sample, said method comprising the steps:
(a) providing a sample comprising genomic template DNA to be tested;
(b) providing a pair of PCR primers selected from the group consisting of: primers comprising at least 15 consecutive bases of the DNA sequence designated SEQ ID NO: 3 or annealing equivalents thereof and SEQ ID NO: 4 or annealing equivalents thereof, or at least 15 consecutive bases of the DNA sequence designated SEQ ID NO: 5 or annealing equivalents thereof and a second primer comprising at least 15 consecutive bases of the DNA sequence designated SEQ ID NO: 6 or an annealing equivalent thereof;
(c) amplifying by means of PCR a region of template DNA using said primer pair to produce one or more PCR products from the said sample, and thereafter analysing the PCR products so as to identify genotype and subgenotype Cryptosporidium in a sample.

5. A method according to claim 4 wherein said sample comprises a faecal sample.

6. A method according to claim 4 wherein genomic template DNA of one or more Cryptosporidium standards of known genotype, and optionally known subgenotype, are also provided and amplified, so as to provide a standard during identification of Cryptosporidium genotype and subgenotype.

7. A method according to claim 4 wherein PCR products are subject to electrophoretic separation

8. A method according to claim 1 wherein said electrophoretic separation comprises denaturing polyacrylamide gel electrophoresis (DGPE) or single-strand conformation polymorphism (SSCP) electrophoresis.

9. An oligonucleotide according to claim 2 wherein said annealing equivalents comprise at least 15 nucleotides in length, and are adapted to anneal to a target sequence which is complementary to at least 15 consecutive bases of a primer selected from SEQ ID NO: 3, SEQ ID NO: 4, SEQ ID NO: 5 or SEQ ID NO: 6 at a temperature of 45 to 55° C. in a buffer containing 1.5-7 mM MgCl₂.
